(19) United States
(12) Patent Application Publication
(10) Pub. No.: US 2025/0019372 A1
(43) Pub. Date: Jan. 16, 2025

(54) CRYSTALLINE FORMS OF A RIPK1 INHIBITOR

(71) Applicant: RIGEL PHARMACEUTICALS, INC., South San Francisco, CA (US)

(72) Inventors: Shaoming DUAN, Foster City, CA (US); Simon SHAW, Oakland, CA (US); Xin WANG, South San Francisco, CA (US)

(21) Appl. No.: 18/711,541

(22) PCT Filed: Dec. 7, 2022
(86) PCT No.: PCT/US2022/052072

§ 371 Date: May 17, 2024

Related U.S. Application Data
(60) Provisional application No. 63/290,066, filed on Dec. 16, 2021.

(51) Int. Cl. C07D 413/12; A61K 31/553

(52) U.S. Cl. CPC C07D 413/12; A61K 31/553

(57) ABSTRACT

The present invention provides a crystalline compound of the formula:

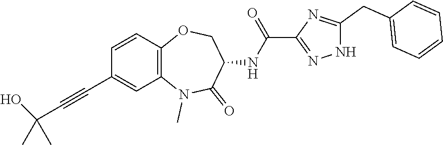 [SMILES: CN1C(=O)[C@@H](NC(=O)c2n[nH]c(Cc3ccccc3)n2)COc2ccc(C#CC(C)(C)O)cc21]

wherein the crystalline compound is useful for treating autoimmune and inflammatory diseases, such as atopic dermatitis, rheumatoid arthritis, and psoriasis.

The present invention relates to certain novel crystalline forms of a receptor-interacting protein-1 kinase (“RIPK1”) inhibitor, (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, to pharmaceutical compositions comprising the crystalline forms, to methods of using the crystalline forms to treat physiological disorders, such as inflammatory and autoimmune diseases, and to processes useful in the synthesis thereof.

RIP1 belongs to the tyrosine kinase-like family and is a serine/threonine protein kinase involved in innate immune signaling. RIP1 plays a central role in regulating cell signaling and its role in programmed cell death has been linked to various autoimmune and inflammatory diseases, such as inflammatory bowel disease, psoriasis, rheumatoid arthritis, and other diseases and/or conditions associated with inflammation and/or necroptotic cell death.

WO 2019/213447 discloses kinase inhibitor compounds, such as RIPK1 inhibitor compounds, useful for treating inflammatory diseases, including for example, the RIPK1 inhibitor, (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide.

Crystalline forms of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide are desired. Furthermore, novel crystalline forms of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide are desired which provide for improved solid state stability. In addition, novel crystalline forms of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide are desired which provide for improved solid state stability for enhanced utilization in the preparation and manufacture of pharmaceutical formulations with improved stability. The present invention provides crystalline compounds that address one or more of these needs.

Accordingly, in one embodiment, the invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline.

In a particular embodiment, the invention further provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is an anhydrate.

In an embodiment, the invention further provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form A characterized by an X-ray powder diffraction (XRPD) pattern using CuKα radiation comprising a peak at diffraction angle 2-theta of 17.5° and one or more peaks at 9.7°, 14.4°, 15.4°, 17.0°, or 17.9°, with a tolerance for the diffraction angles of ±0.2 degrees.

In a further embodiment, the invention provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form B characterized by an XRPD pattern using CuKα radiation comprising a peak at diffraction angle 2-theta of 18.1° and one or more peaks at 9.9°, 11.5°, 12.2°, 14.7°, or 16.5°, with a tolerance for the diffraction angles of ±0.2 degrees.

In a further embodiment, the invention provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide monoethanol solvate Form C characterized by an XRPD pattern using CuKα radiation comprising a peak at diffraction angle 2-theta of 6.8° and one or more peaks at 4.9°, 9.9°, 13.6°, or 18.4°, with a tolerance for the diffraction angles of ±0.2 degrees.

In a particular embodiment, the invention provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D characterized by at least one of the following:
a) an X-ray powder diffraction pattern using CuKα radiation comprising a peak at diffraction angle 2-theta of 7.0° and one or more peaks at 11.2°, 17.4°, or 19.5°, with a tolerance for the diffraction angles of ±0.2 degrees; and
b) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

In an embodiment, the invention provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D characterized by at least one of the following:
c) an X-ray powder diffraction pattern using CuKα radiation comprising a peak at diffraction angle 2-theta of 7.0° and one or more peaks at 11.2°, 16.9°, 17.4°, or 19.5°, with a tolerance for the diffraction angles of ±0.2 degrees; and
d) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

In an embodiment, the invention provides crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D characterized by at least one of the following:
e) an X-ray powder diffraction pattern using CuKα radiation comprising a peak at diffraction angle 2-theta of 7.0° and one or more peaks at 11.2°, 15.1°, 16.9°, 17.4°, or 19.5°, with a tolerance for the diffraction angles of ±0.2 degrees; and
f) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

In an embodiment, the present invention further provides a method of treating an inflammatory disease in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention further provides a method of treating an autoimmune disease in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention further provides a method of treating atopic dermatitis in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention further provides a method of treating inflammatory bowel disease in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention further provides a method of treating rheumatoid arthritis in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention also provides a method of treating psoriasis in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention also provides a method of treating systemic lupus erythematosus in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention also provides a method of treating gout in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention also provides a method of treating cutaneous lupus erythematosus in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline. In an embodiment, the present invention also provides a method of treating lupus nephritis in a patient in need of such treatment, comprising administering to the patient an effective amount of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline.

In one embodiment of the method of treatments set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is a crystalline anhydrate. In a further embodiment of the method of treatments set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is Form D crystalline anhydrate.

In an embodiment, the present invention further provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in therapy. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating an inflammatory disease. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating an autoimmune disease. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating atopic dermatitis. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating rheumatoid arthritis. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating inflammatory bowel disease. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating psoriasis. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating systemic lupus erythematosus. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating gout. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating cutaneous lupus erythematosus. In an embodiment, the present invention provides (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for use in treating lupus nephritis.

In one embodiment of the therapeutic uses set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is a crystalline anhydrate. In a further embodiment of the therapeutic uses set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is Form D crystalline anhydrate.

In an embodiment, the present invention also provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating an inflammatory disease. In an embodiment, the present invention provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating an autoimmune disease. In an embodiment, the present invention provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating atopic dermatitis. In an embodiment, the present invention provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating rheumatoid arthritis. In an embodiment, the present invention further provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating inflammatory bowel disease. In an embodiment, the present invention further provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating psoriasis. In an embodiment, the present invention also provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating systemic lupus erythematosus. In an embodiment, the present invention also provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating gout. In an embodiment, the present invention also provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating cutaneous lupus erythematosus. In an embodiment, the present invention also provides the use of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline for the manufacture of a medicament for treating lupus nephritis.

In one embodiment of the medicaments set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is a crystalline anhydrate. In a further embodiment of the medicaments set out above, the crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide is Form D crystalline anhydrate.

In an embodiment, the present invention further provides a pharmaceutical composition, comprising (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline with one or more pharmaceutically acceptable carriers, diluents, or excipients. In an embodiment, the present invention further provides a pharmaceutical composition, comprising (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is a crystalline anhydrate with one or more pharmaceutically acceptable carriers, diluents, or excipients. In an embodiment, the present invention further provides a process for preparing a pharmaceutical composition, comprising admixing (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which is crystalline with one or more pharmaceutically acceptable carriers, diluents, or excipients. In an embodiment, the present invention also encompasses processes for the synthesis of (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide which are crystalline.

As used herein, the terms “treating”, “treatment”, or “to treat” includes restraining, slowing, stopping, or reversing the progression or severity of an existing symptom or disorder.

As used herein, the term “patient” refers to a mammal, in particular a human.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of the present invention are preferably formulated as pharmaceutical compositions administered by any route which makes the compound bioavailable, including oral, transdermal, and parenteral routes. Most preferably, such compositions are for oral administration. Such pharmaceutical compositions and processes for preparing same are well known in the art. (See, e.g., Remington: The Science and Practice of Pharmacy, A. Adejare, Editor, 23^{nd} Edition, published 2020, Elsevier Science).

Certain abbreviations are defined as follows: “MIBK” refers to methyl isobutyl ketone; “EtOAc” refers to ethyl acetate; “EtOH” refers to ethanol; “DMSO” refers to dimethyl sulfoxide; “RT” refers to room temperature; “HPLC” refers to high performance liquid chromatography; “XRPD” refers to X-ray powder diffraction; “mL” refers to milliliter or milliliters; “nm” refers to nonometer or nanometers; “rpm” refers to revolutions per minute; and “min” refers to minute or minutes.

The name “(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide,” corresponds to the structure of Formula I:

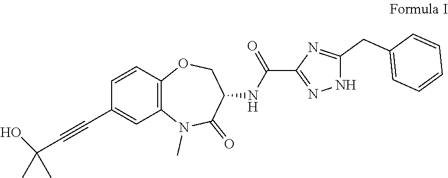 [SMILES: CN1C(=O)[C@@H](NC(=O)c2n[nH]c(Cc3ccccc3)n2)COc2ccc(C#CC(C)(C)O)cc21]

The amorphous form of the compound of Formula I, (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, may be prepared by procedures known to one of ordinary skill in the art such as that taught in WO 2019/213447. The following Examples further illustrate the invention.

### EXAMPLE 1

### Preparation of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form D

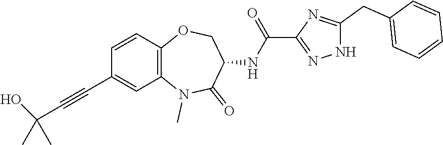 [SMILES: CN1C(=O)[C@@H](NC(=O)c2n[nH]c(Cc3ccccc3)n2)COc2ccc(C#CC(C)(C)O)cc21]

Amorphous (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide (316.3 mg) was suspended in a mixture of acetone/n-heptane (1:2, v/v, 3 mL). The suspension was stirred at RT for about 3 days. The solids were isolated by centrifugation (10,000 rpm, 2 minutes) and then dried under vacuum at RT for about 1 day to provide the title compound. The crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D was found to be the most thermodynamically stable crystalline form compared to the other identified Forms A, B, and C at a temperature range of RT to 50° C.

### EXAMPLE 2

### Preparation of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form A

Amorphous (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide (about 15 mg) was suspended in MIBK/n-heptane (1:1, v/v, 0.3 mL) in an HPLC vial. After the suspension was stirred magnetically for 4 days at RT, the remaining solids were isolated to provide the title compound.

### EXAMPLE 3

### Preparation of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form B

Amorphous (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide (about 15 mg) was suspended in EtOAc/n-heptane (1:1, v/v, 0.3 mL) in an HPLC vial. After the suspension was stirred magnetically for 4 days at RT, the remaining solids were isolated to provide the title compound.

### EXAMPLE 4

### Preparation of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Monoethanol Solvate, Form C

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Form D (53.0 mg) was dissolved in a mixture of EtOH/H₂O (1:1, v/v, 3 mL) into a clear solution at ˜75° C. The solution was hot-filtered through a pre-warmed syringe filter (0.45 μm GHP) into a clean vial and cooled to 2-8° C. in 3 steps (44° C., RT, and then placed into a refrigerator). Aggregates of needles were produced in the solution after the sample was kept at 2-8° C. for 5 days, and isolated by decanting the liquid to provide the title compound. The wet solids were analyzed by XRPD.

### X-Ray Powder Diffraction Conditions for Examples 1-3

The XRPD analysis was carried out on a PANalytical X'Pert3 X-ray powder diffractometer. The XRPD measurement conditions used are listed in Table 1.

TABLE 1
Measurement conditions for XRPD analysis

| | |
|---|---|
| Scan Axis | Gonio |
| Start Position (°2θ) | 3.0131 |
| End Position (°2θ) | 39.9851 |
| Step Size (°2θ) | 0.0260 |
| Scan Step Time (s) | 46.6650 |
| Scan Type | Continuous |
| PSD Mode | Scanning |
| PSD Length (°2θ) | 3.35 |
| Offset (°2θ) | 0.0000 |
| Divergence slit type | Fixed |
| Divergence slit size (°) | 0.1089 |
| Specimen Length (mm) | 10.00 |
| Measurement Temperature (° C.) | 25 |
| Anode Material | Cu |
| Kα1 (Å) | 1.54060 |
| Kα2 (Å) | 1.54443 |
| Kβ (Å) | 1.39225 |
| Kα2/Kα1 | 0.5000 |
| X-Ray tube Setting | 45 kV, 40 mA |
| Goniometer Radius (mm) | 240.00 |
| Dist. Focus-Diverg. Slit (mm) | 100.00 |
| Incident Beam Monochromator | No |
| Spinning | Yes |

### Table 2. XRPD Peaks of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form D

| | | Relative Intensity |
|---|---|---|
| | Angle (°2- | (% of most intense |
| Peak | Theta) +/−0.2° | peak) |
| 1 | 7.0 | 100 |
| 2 | 11.2 | 20.1 |
| 3 | 13.3 | 8.5 |
| 4 | 14.0 | 5.6 |
| 5 | 15.1 | 63.5 |
| 6 | 16.9 | 66.3 |
| 7 | 17.4 | 35.4 |
| 8 | 18.2 | 16.7 |
| 9 | 18.9 | 7.9 |
| 10 | 19.5 | 89.1 |
| 11 | 19.9 | 8.6 |
| 12 | 20.3 | 30.8 |
| 13 | 20.7 | 20.1 |
| 14 | 21.2 | 33.5 |
| 15 | 21.8 | 20.1 |
| 16 | 22.3 | 13.9 |
| 17 | 22.6 | 40.5 |
| 18 | 23.3 | 17.9 |
| 19 | 25.9 | 14.4 |
| 20 | 26.7 | 26.1 |
| 21 | 27.8 | 16.7 |
| 22 | 31.4 | 9.9 |
| 23 | 32.8 | 5.6 |

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D is characterized by an XRPD pattern using CuKα radiation as having diffraction peaks (2-theta values) as described in Table 2, and in particular comprising a peak at diffraction angle 2-theta of 7.0° and one or more peaks at 11.2°, 17.4°, or 19.5°, with a tolerance for the diffraction angles of ±0.2 degrees.

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D is further characterized by an XRPD pattern using CuKα radiation as having diffraction peaks (2-theta values) as described in Table 2, and in particular comprising a peak at diffraction angle 2-theta of 7.0° and one or more peaks at 11.2°, 16.9°, 17.4°, or 19.5°, with a tolerance for the diffraction angles of ±0.2 degrees.

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D is further characterized by an XRPD pattern using CuKα radiation as having diffraction peaks (2-theta values) as described in Table 2, and in particular comprising a peak at diffraction angle 2-theta of 7.0° and one or more peaks at 11.2°, 15.1°, 16.9°, 17.4°, or 19.5°, with a tolerance for the diffraction angles of ±0.2 degrees.

¹³C Solid State NMR (¹³C ssNMR)

Without weighing, a small amount of material was scooped into a 4 mm rotor and compacted to ensure an even distribution. This was repeated until the rotor was filled. The ¹³C ssNMR for crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form D was acquired at ambient temperature on a Bruker Avance III HD with a Bruker Ultrashield 400WB Plus magnet operating at a frequency of 100.6 MHz. The probe employed was Bruker MAS 4 BL CP BB DVT N-P/H. Acquisitional parameters were as follows: 15552 scans, 34 ms acquisition time, 2.5 s interpulse delay, 10 kHz MAS frequency, 1.5 ms contact time, and a SPINAL64 decoupling scheme. The data were externally referenced to adamantane at 29.5 ppm.

Representative ¹³C ssNMR resonances for for crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate, Form D include: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

### Table 3. XRPD Peaks of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form A

| | | Relative Intensity |
|---|---|---|
| | Angle (°2- | (% of most intense |
| Peak | Theta) +/−0.2° | peak) |
| 1 | 9.7 | 65.1 |
| 2 | 10.8 | 37.8 |
| 3 | 14.4 | 16.4 |
| 4 | 15.1 | 32.5 |
| 5 | 15.4 | 34.4 |
| 6 | 15.8 | 35.5 |
| 7 | 17.0 | 94.9 |
| 8 | 17.5 | 100.0 |
| 9 | 17.9 | 84.9 |
| 10 | 18.9 | 21.5 |
| 11 | 19.4 | 46.6 |
| 12 | 20.4 | 68.3 |
| 13 | 21.3 | 13.1 |
| 14 | 21.9 | 17.5 |
| 15 | 22.3 | 44.8 |
| 16 | 22.7 | 34.8 |
| 17 | 23.4 | 17.2 |
| 18 | 24.0 | 34.3 |
| 19 | 24.5 | 19.9 |
| 20 | 25.4 | 12.1 |

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate Form A is characterized by an XRPD pattern using CuKα radiation as having diffraction peaks (2-theta values) as described in Table 3, and in particular comprising a peak at diffraction angle 2-theta of 17.5° and one or more peaks at 9.7°, 14.4°, 15.4°, 17.0°, or 17.9°, with a tolerance for the diffraction angles of ±0.2 degrees.

### Table 4. XRPD Peaks of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Anhydrate, Form B

| | | Relative Intensity |
|---|---|---|
| | Angle (°2- | (% of most intense |
| Peak | Theta) +/−0.2° | peak) |
| 1 | 5.7 | 6.4 |
| 2 | 9.9 | 22.0 |
| 3 | 10.3 | 12.2 |
| 4 | 10.8 | 4.1 |
| 5 | 11.5 | 13.4 |
| 6 | 12.2 | 16.5 |
| 7 | 13.1 | 15.5 |
| 8 | 14.1 | 16.2 |
| 9 | 14.7 | 17.4 |
| 10 | 15.2 | 48.3 |
| 11 | 15.8 | 2.9 |
| 12 | 16.5 | 18.7 |
| 13 | 17.3 | 96.2 |
| 14 | 18.1 | 100.0 |
| 15 | 18.7 | 41.7 |
| 16 | 19.1 | 11.8 |
| 17 | 19.9 | 20.7 |
| 18 | 20.1 | 15.9 |
| 19 | 21.2 | 63.0 |
| 20 | 22.3 | 34.4 |
| 21 | 22.6 | 19.7 |
| 22 | 23.1 | 29.0 |
| 23 | 23.4 | 26.6 |
| 24 | 23.8 | 17.8 |
| 25 | 24.4 | 5.6 |
| 26 | 25.1 | 17.1 |

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide anhydrate, Form B is characterized by an XRPD pattern using CuKα radiation as having diffraction peaks (2-theta values) as described in Table 4, and in particular comprising a peak at diffraction angle 2-theta of 18.1° and one or more peaks at 9.9°, 11.5°, 12.2°, 14.7°, or 16.5° with a tolerance for the diffraction angles of ±0.2 degrees.

### XRPD Conditions for Example 4

The XRPD pattern of crystalline solids was obtained on a Bruker D8 Endeavor X-ray powder diffractometer, equipped with a CuKα (1.5418 Å) source and a Linxeye detector, operating at 40 kV and 40 mA. The sample is scanned between 4 and 42 2θ°, with a step size of 0.009 2θ° and a scan rate of 0.5 seconds/step, and using 0.3° primary slit opening, and 3.9° PSD opening. The powder is packed wet on a quartz sample holder and a smooth surface is obtained using a glass slide. The crystal form diffraction patterns are collected at ambient temperature and relative humidity. Crystal peak positions are determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the crystalline forms are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### Table 5. XRPD Peaks of Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide Monoethanol Solvate, Form C

| | | Relative Intensity |
|---|---|---|
| | Angle (°2- | (% of most intense |
| Peak | Theta) +/−0.2° | peak) |
| 1 | 4.9 | 12.3 |
| 2 | 6.8 | 5.0 |
| 3 | 9.5 | 1.1 |
| 4 | 9.9 | 34.9 |
| 5 | 10.7 | 3.1 |
| 6 | 13.6 | 8.1 |
| 7 | 15.0 | 18.6 |
| 8 | 15.8 | 0.3 |
| 9 | 16.9 | 0.6 |
| 10 | 17.8 | 3.1 |
| 11 | 18.4 | 2.9 |
| 12 | 19.1 | 3.4 |
| 13 | 19.7 | 1.5 |
| 14 | 20.1 | 100.0 |
| 15 | 20.2 | 26.4 |
| 16 | 21.5 | 1.5 |
| 17 | 22.2 | 1.1 |
| 18 | 22.6 | 1.1 |
| 19 | 22.9 | 7.6 |
| 20 | 23.9 | 1.3 |
| 21 | 25.2 | 8.3 |
| 22 | 25.4 | 3.0 |

Crystalline (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide monoethanol solvate, Form C is characterized by an XRPD pattern using CuKα radiation as having diffraction peaks (2-theta values) as described in Table 5, and in particular comprising a peak at diffraction angle 2-theta of 6.8° and one or more peaks at 4.9°, 9.9 °, 13.6°, or 18.4° with a tolerance for the diffraction angles of ±0.2 degrees.

What is claimed is:

1. A crystalline form of a compound of the formula:

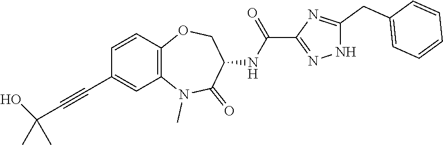 [SMILES: CN1C(=O)[C@@H](NC(=O)c2n[nH]c(Cc3ccccc3)n2)COc2ccc(C#CC(C)(C)O)cc21]

2. The crystalline form of claim 1, which is a crystalline anhydrate.

3. The crystalline form of claim 2, which is characterized by at least one of the following:
a) an X-ray powder diffraction pattern using CuKα radiation comprising a peak at diffraction angle 2-theta of 7.0° and one or more peaks at 11.2°, 17.4°, or 19.5°, with a tolerance for the diffraction angles of ±0.2 degrees; and
b) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

4. The crystalline form of claim 2, which is characterized by at least one of the following:
a) an X-ray powder diffraction pattern using CuKα radiation comprising a peak at diffraction angle 2-theta of 7.0° and one or more peaks at 11.2°, 16.9°, 17.4°, or 19.5°, with a tolerance for the diffraction angles of ±0.2 degrees and
b) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

5. The crystalline form of claim 2, which is characterized by at least one of the following:
a) an X-ray powder diffraction pattern using CuKα radiation comprising a peak at diffraction angle 2-theta of 7.0° and one or more peaks at 11.2°, 15.1°, 16.9°, 17.4°, or 19.5°, with a tolerance for the diffraction angles of ±0.2 degrees; and
b) a ¹³C solid state NMR spectrum which comprises peaks referenced to the highfield resonance of adamantane (δ=29.5 ppm) at: 168.5, 159.7, 157.4, 156.0, 149.5, 138.5, 136.6, 129.7, 129.1, 127.5, 126.6, 123.5, 121.9, 98.5, 79.8, 77.2, 65.8, 49.1, 36.4, 34.9, 32.5, and 30.5 ppm (±0.2 ppm respectively).

6. The crystalline form according to claim 2 characterized by an X-ray powder diffraction pattern using CuKα radiation comprising a peak at diffraction angle 2-theta of 17.5° and one or more peaks at 9.7°, 14.4°, 15.4°, 17.0°, or 17.9°, with a tolerance for the diffraction angles of ±0.2 degrees.

7. The crystalline form according to claim 2 characterized by an X-ray powder diffraction pattern using CuKα radiation comprising a peak at diffraction angle 2-theta of 18.1° and one or more peaks at 9.9°, 11.5°, 12.2°, 14.7°, or 16.5°, with a tolerance for the diffraction angles of ±0.2 degrees.

8. A method of treating an inflammatory disease in a patient, comprising administering to a patient in need of such treatment an effective amount of a crystalline form according to claim 3.

9. A method of treating an autoimmune disease in a patient, comprising administering to a patient in need of such treatment an effective amount of a crystalline form according to claim 3.

10. A method of treating psoriasis in a patient, comprising administering to a patient in need of such treatment an effective amount of a crystalline form according to claim 3.

11. A method of treating atopic dermatitis in a patient, comprising administering to a patient in need of such treatment an effective amount of a crystalline form according to claim 3.

12. A method of treating rheumatoid arthritis in a patient, comprising administering to a patient in need of such treatment an effective amount of a crystalline form according to claim 3.

13-23. (canceled)

24. A pharmaceutical composition, comprising a crystalline form according to claim 3 with one or more pharmaceutically acceptable carriers, diluents, or excipients.

25. (canceled)

26. A method of treating an inflammatory bowel disease in a patient, comprising administering to a patient in need of such treatment an effective amount of a crystalline form according to claim 3.

27. A method of treating an inflammatory or autoimmune disease in a patient, comprising administering to a patient in need of such treatment an effective amount of a crystalline form according to claim 6.

28. A method of treating psoriasis, atopic dermatitis, rheumatoid arthritis, or inflammatory bowel disease in a patient, comprising administering to a patient in need of such treatment an effective amount of a crystalline form according to claim 6.

29. A method of treating an inflammatory or autoimmune disease in a patient, comprising administering to a patient in need of such treatment an effective amount of a crystalline form according to claim 7.

30. A method of treating psoriasis, atopic dermatitis, rheumatoid arthritis, or inflammatory bowel disease in a patient, comprising administering to a patient in need of such treatment an effective amount of a crystalline form according to claim 7.

31. A method of treating an inflammatory or autoimmune disease in a patient, comprising administering to a patient in need of such treatment an effective amount of a crystalline form according to claim 5.

32. A method of treating psoriasis, atopic dermatitis, rheumatoid arthritis, or inflammatory bowel disease in a patient, comprising administering to a patient in need of such treatment an effective amount of a crystalline form according to claim 5.
